(19) United States
(12) Patent Application Publication
(10) Pub. No.: US 2016/0199127 A1
(43) Pub. Date: Jul. 14, 2016

(54) METHODS AND APPARATUSES FOR TREATING AUTO-IMMUNE DISEASES BY ABLATIVE NEUROMODULATION

(71) Applicant: La Vita Technologies Ltd., Hamilton (BM)

(72) Inventor: David Prutchi, Voorhees, NJ (US)

(21) Appl. No.: 14/912,089

(22) PCT Filed: Aug. 14, 2014
(86) PCT No.: PCT/IB2014/063924

§ 371 Date: Feb. 14, 2016

Related U.S. Application Data
(60) Provisional application No. 61/865,636, filed on Aug. 14, 2013.

(51) Int. Cl. A61B 18/14; A61B 17/221

(52) U.S. Cl. CPC A61B 18/1492; A61B 17/221; A61B 2018/00267

(57) ABSTRACT

The present invention, in some embodiments thereof, relates to intravascular neural ablation and, more particularly, but not exclusively, to tools and methodologies for treating systemic nerve hyperactivity through splenic and/or carotid denervation. Devices are disclosed for performing ablation and protecting a patient from formation of embolisms. Furthermore a branching ablation unit is disclosed.

### RELATED APPLICATION/S

This application claims the benefit of priority under 35 USC §119(e) of U.S. Provisional Patent Application No. 61/865,636 filed 14 Aug. 2013, the contents of which are incorporated herein by reference in their entirety.

### FIELD AND BACKGROUND OF THE INVENTION

The present invention, in some embodiments thereof, relates to intravascular neural ablation and, more particularly, but not exclusively, to tools and methodologies for treating systemic nerve hyperactivity through splenic and/or carotid denervation.

U.S. Pat. No. 7,766,960 discloses a delivery catheter for use in deploying a vascular prosthesis having a self-expanding helical section.

U.S. Pat. No. 5,383,856 discloses a balloon catheter device designed to be especially well suited to repair or tack dissections in a blood vessel, and a method for repairing dissections.

International patent publication WO2014/118733 discloses an ablation device and/or method of ablation including placing one or more ablation electrodes in contact with a target tissue in a lumen.

International patent publication WO2014/118785 discloses an ablation device and/or method of ablation including placing one or more ablation electrodes in contact with a target tissue in a lumen.

Additional background art includes: Bakhiet M, Yu L Y, Ozenci V, Khan A, Shi F D, “Modulation of immune responses and suppression of experimental autoimmune myasthenia gravis by surgical denervation of the spleen”, Clin Exp Immunol., 144(2):290-8, 2006; Boyle D L, Edgar M, Sorkin L, Firestein G S, “Role of the Central Nervous System (CNS) in Peripheral Inflammation: Sympathetic Innervation of the Spleen Regulates Inflammatory Arthritis.” Arthritis & Rheumatism, Volume 62, November 2010 Abstract Supplement, Abstracts of the American College of Rheumatology/Association of Rheumatology Health Professionals Annual Scientific Meeting, Atlanta, Ga., Nov. 6-11, 2010; Buijs R M, van der Vliet J, Garidou M-L, Huitinga I, Escobar C, “Spleen Vagal Denervation Inhibits the Production of Antibodies to Circulating Antigens.” PLoS ONE 3(9): e3152. doi:10.1371/journal.pone.0003152, 2008; Gelfand M, Levin H, Method for sympathetic rebalancing of patient, US 20120172680 A1, 2012; Rasouli J, Lekhraj R, Ozbalik M, Lalezari P, Casper D, “Brain-Spleen Inflammatory Coupling: A Literature Review”, Einstein J Biol Med.; 27(2): 74-77, 2011; Rosas-Ballina M, Olofsson P S, Ochani M, Valdés-Ferrer S I, Levine Y A, Reardon C A, Tusche M W, Pavlov V A, Andersson U, Chavan S, Mak T W, Tracey K J, “Acetylcholine-Synthesizing T Cells Relay Neural Signals in a Vagus Nerve Circuit”, Science 7 Oct. 2011: Vol. 334 no. 6052 pp. 98-101, 2011.

### SUMMARY OF THE INVENTION

According to an aspect of some embodiments of the present invention there is provided a tool for ablation of tissue in a living patient comprising: a plurality of ablation electrodes; a basket mounted axially to a shaft, the basket having a radially contracted configuration wherein supports of the basket are oriented along an axis of the basket for fitting into a channel of a catheter, a distal end of the catheter fitting into a lumen of the living patient and a radially spread configuration wherein the supports are spread radially away from the axis for holding the plurality of electrodes against an inner wall of the lumen; a cup shaped embolic trap configured to spread to block the lumen to transport of emboli, the embolic trap spreading radially around an apex located along an axis of the basket and distal to the basket; and a manipulation apparatus configured to be accessible from the proximal end of the catheter the manipulation apparatus configured for reversibly extending and retrieving the shaft including the basket and the plurality of electrodes and the embolic trap through a distal opening of the catheter and reversibly switching the basket between the radially contracted configuration and the radially spread configuration.

According to some embodiments of the invention, the embolic trap is mounted to the shaft, distal to the basket.

According to some embodiments of the invention, the embolic trap is mounted to a distal end of the basket.

According to some embodiments of the invention, the plurality of ablation electrodes, the embolic trap and the basket fit concurrently into the channel.

According to some embodiments of the invention, a distance between the basket and the trap along the axis of the channel is fixed.

According to some embodiments of the invention, embolic trap also has a radially spread and a radially contracted configuration and where the manipulation apparatus is further configured for reversibly switching the embolic trap between a radially spread and a radially contracted configuration.

According to some embodiments of the invention, basket is spread and contracted independently from the embolic trap.

According to some embodiments of the invention, manipulation apparatus spreads the basket only when the embolic trap is in the radially spread configuration.

According to some embodiments of the invention, the basket and the embolic trap have three stages of deployment: a fully retracted state wherein both the embolic trap and basket are radially contracted; an intermediate state wherein the embolic trap radially spread and the basket is radially contracted and a fully expanded state wherein the embolic trap and basket are radially expended.

According to some embodiments of the invention, the tool further includes one or more sensors configured to detect a slew rate and/or propagation time between two electrodes, the two electrodes being selected from the plurality of ablation electrodes and a dispersive electrode.

According to some embodiments of the invention, the tool further includes a dispersive electrode having a surface area of electrical contact at least ten times the surface area of electrical contact of at least one electrode of the plurality of ablation electrodes.

According to some embodiments of the invention, a distal end of the dispersive electrode is located at least 5 mm proximal from the most proximal electrode of the plurality of ablation electrodes.

According to some embodiments of the invention, a distal end of the dispersive electrode is located less than 100 mm proximal from most proximal electrode of the plurality of ablation electrodes.

According to some embodiments of the invention, the tool further includes an insulator electrically insulating at least one of the plurality of ablation electrodes from a fluid in the lumen.

According to some embodiments of the invention, the tool further includes one or more sensors detecting an indicator of ablation progress; and a control unit programmed to: receive from the one or more sensors an indicator of progress of a bipolar ablation process between a pair of the plurality of ablation electrodes, identify a zone for further ablation based on the received indicator, and instruct to ablate the zone with a unipolar signal between the dispersive electrode and at least one of the plurality of ablation electrodes.

According to some embodiments of the invention, the one or more sensors detect a slew and/or propagation time between two electrodes selected from the plurality of ablation electrodes and the dispersive electrode.

According to an aspect of some embodiments of the present invention there is provided a system for determining progress of denervation of a lumen located in a living patient, comprising: a sheath, a distal end of the sheath for insertion into the lumen, a plurality of ablation electrodes; a basket mounted axially to a shaft, the basket having a radially contracted configuration wherein supports of the basket are oriented along an axis of the basket for fitting into a channel of a catheter, a distal end of the catheter fitting into the lumen and a radially spread configuration wherein the supports are spread radially away from the axis for holding the plurality of electrodes against an inner wall of the lumen; a manipulation apparatus configured to be accessible from the proximal end of the catheter the manipulation apparatus configured for reversibly extending and retrieving the basket and the plurality of electrodes through a distal opening of the sheath and reversibly switching the basket between the radially contracted configuration and the radially spread configuration; and a control unit configured to detect a parameter selected from the group consisting of a slew rate and propagation time between at least one pair of the plurality of ablations electrodes.

According to some embodiments of the invention, the system further includes an embolic trap configured for blocking transport of emboli in the lumen and wherein the manipulation apparatus is further configured for reversibly extending and retrieving the embolic trap through a distal opening of the sheath.

According to an aspect of some embodiments of the present invention there is provided an ablation device including: a plurality of pairs of ablation electrodes arranged along a single shaft; the single shaft having at least two configurations: a longitudinally stretched configuration wherein the plurality of pairs of ablation electrodes are arranged linearly for insertion into a channel of a catheter fitting into a lumen, and a radially spread configuration wherein the single shaft is bent into a helix that is circumscribed by and in contact with an inner wall of the lumen and retains the plurality of pairs of ablation electrodes in a predetermined pattern along the inner wall of the lumen; and a manipulation mechanism accessible from outside the lumen, the manipulation mechanism for longitudinally contracting the single shaft inside the lumen from the stretched configuration to the radially spread configuration.

According to some embodiments of the invention, a proximal end of the shaft is connected to a catheter extending out of the lumen.

According to some embodiments of the invention, a proximal end of the helix is centered along the lumen.

According to an aspect of some embodiments of the present invention there is provided an ablation catheter including: a stem including a junction at a distal end thereof; a plurality of branches extending from the junction, each of the plurality of branches including a plurality of electrodes; and a control unit configured for transmitting a radio frequency ablation signal between at least one of the plurality of electrodes of a first branch of the plurality of branches to at least one electrode of the plurality of electrodes on a second branch one of the plurality of branches.

According to some embodiments of the invention, at least one of the plurality of branches is retractable.

According to some embodiments of the invention, a distance between the junction and a distal end of at least one of the plurality of branches is between 10 to 50 mm from the junction.

According to some embodiments of the invention, a distance between the at least one electrode and the junction is between 3 to 20 mm.

According to some embodiments of the invention, a width of the stem is less than 9 Fr.

According to some embodiments of the invention, a width of the stem is less than 6 Fr.

According to an aspect of some embodiments of the present invention there is provided a method of treatment of an inflammatory autoimmune disease including: inserting a plurality of pairs of electrodes into a splenic artery; arranging the plurality of pairs of electrodes in a predetermined pattern along a wall of the splenic artery; activating the electrodes to ablate a sympathetic nerve by radio frequency ablation; returning the plurality of pairs of electrodes out of the splenic artery.

According to some embodiments of the invention, the activating includes applying a radiofrequency signal of power between 2 to 10 Watts to the sympathetic nerve.

According to some embodiments of the invention, the activating includes forming multiple lesions having a predetermined geometry on a wall of the splenic artery.

According to some embodiments of the invention, the sympathetic nerve includes at least one structure selected from a nerve located in an adventitia of the splenic artery, a ganglia located close to the splenic artery, an area in proximity to a ostium of the spleen, an area in proximity with an aorta.

According to an aspect of some embodiments of the present invention there is provided a method of treatment of an inflammatory autoimmune disease comprising: Inserting a plurality of pairs of ablation electrodes into a common carotid artery; arranging the plurality of pairs of ablation electrodes in a predetermined pattern along a wall of one or more of the common carotid artery, an external carotid artery and an internal carotid artery; activating at least one pair of the multiple pairs of ablation electrodes to ablate a sympathetic nerve by radio frequency ablation; and returning the plurality of pairs of electrodes out of the common carotid artery.

According to some embodiments of the invention, the activating includes applying a radiofrequency signal of power between 2 to 10 Watts to the sympathetic nerve.

According to some embodiments of the invention, the activating includes forming multiple lesions having a predetermined geometry the wall.

According to some embodiments of the invention, the method further includes inserting a first electrode of the plurality of pairs of ablation electrodes into an external carotid artery; and transmitting a radio frequency signal between the first electrode and a second electrode of the plurality of pairs of ablation electrodes located outside the external carotid artery.

According to some embodiments of the invention, the second electrode is located in an inner carotid artery.

According to some embodiments of the invention, the method further includes applying a unifying force between the first electrode and the second electrode.

According to some embodiments of the invention, the applying includes applying a magnetic force.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart illustrating a method of ablating tissue with embolic protection in accordance with an embodiment of the current invention;

FIG. 2 is a flowchart illustrating a method of ablating tissue with a branching catheter in accordance with an embodiment of the current invention;

FIG. 3 is a flowchart illustrating a method of evaluating progress of ablation in accordance with an embodiment of the current invention;

FIGS. 4A-C illustrate an ablation tool with separate insulation and embolic protection in accordance with an embodiment of the current invention;

FIGS. 5A-B illustrate a tool catheter with integral insulation and embolic protection in accordance with an embodiment of the current invention;

FIG. 6 illustrates a cross section of a catheter channel for transporting an ablation tool in accordance with an embodiment of the current invention;

FIGS. 7A-E illustrate deployment and retrieval of an ablation catheter with an embolic trap in a lumen in accordance with an embodiment of the current invention;

FIGS. 8A-C illustrate a single shaft ablation unit in accordance with an embodiment of the current invention;

FIGS. 9A-C illustrate a manipulation apparatus for an ablation tool in accordance with an embodiment of the current invention;

FIG. 10 illustrates ablation of a carotid body with embolic protection in accordance with an embodiment of the current invention;

FIG. 11 illustrates ablation of a carotid body with a branching catheter in accordance with an embodiment of the current invention; and

FIG. 12 illustrates a branching catheter in accordance with an embodiment of the current invention.

### DESCRIPTION OF SPECIFIC EMBODIMENTS OF THE INVENTION

The present invention, in some embodiments thereof, relates to intravascular neural ablation and, more particularly, but not exclusively, to tools and methodologies for treating systemic nerve hyperactivity through splenic and/or carotid denervation.

### Overview

An aspect of some embodiments of the current invention relates to a tool including a radio ablation unit and an embolic trap mounted along a single shaft for deployment, retrieval and redeployment from a single channel of a catheter while the catheter remains inserted into a lumen of a patient. Optionally a manipulation apparatus located at a proximal end of the catheter controls deployment and/or functioning of both the embolic trap and the ablation unit. The tool may include ablation electrodes, a support structure for positioning the electrodes and a trap for embolic particles.

Optionally, the tool may have multiple states. For example, an operator at a proximal end of a catheter may switch the tool located at the distal end of the catheter from one state to another. For example the states of the tool may include the following:
a fully contracted state wherein both the ablation unit and the embolic trap are contracted—for example in the fully contracted state the ablation unit and the embolic trap may fit together into a catheter channel;
an intermediate state in which the embolic trap is spread radially to contact the inner walls of a lumen to block embolic particles from being transported through the lumen while the ablation unit is at least partially contracted away from the walls of the lumen, and/or
a fully expanded state wherein both the embolic trap and the ablation unit are spread radially: for example the ablation unit is spread to contact the walls of a lumen for performing an ablation and the embolic trap is spread radially to contact the walls of the lumen and/or to block transport of embolic particles through the lumen.

Optionally a manipulation apparatus may be configured to control extension of the tool out from the catheter channel and/or retrieval of the tool back to the channel and/or switching the tool between states. Optionally expansion of the ablation unit and the embolic trap may be by a single mechanical unit. Alternatively or additionally expansion of the ablation unit and the embolic trap may be by or separate mechanical units. For example a single mechanical unit may spread and/or contract the ablation unit and the embolic trap together. Alternatively or additionally a single mechanical unit may spread and/or contract the ablation unit and the embolic trap according to a predetermined sequence. Alternatively or additionally separate mechanical units may allow an operator to spread and/or contract the ablation unit and the embolic trap independently.

Optionally, the ablation unit and the embolic trap are connected to a single shaft. For example, the shaft may be used to extend the ablation unit and the embolic trap together out of a distal end of the catheter. For example the trap and/or the electrodes may be arranged at a fixed longitudinal distance one from the other. For example an apex of the embolic trap may be fixed to a distal end of the basket and/or at a distal distance ranging for example between 0 mm to 10 mm and/or between 10 mm to 50 mm from the distal end of the basket. Alternatively or additionally the trap and/or the electrodes may be extended out of the distal end of the catheter independently.

Optionally, an operator inserts a distal end of a catheter into a lumen to a treatment location. The operator may use a single shaft and/or manipulation apparatus to extend the tool (including for example the ablation unit and the embolic trap) into the lumen. Optionally the tool may be used in the lumen to perform ablation therapy. After performing an ablation, the user may contract the tool, and/or return the tool to the catheter. Without removing the catheter from the patient the operator may further move the catheter and/or deploy the tool (including the ablation unit and the embolic trap) in a new location and/or perform further therapy in the new location.

Alternatively or additionally the operator may remove the tool from the patient without removing the catheter and/or without removing a guidewire from the patient.

In some embodiments the ablation unit and/or the embolic trap may be deployed according to a predetermined sequence. Optionally, the embolic trap is deployed before the ablation unit, for example to prevent transport of emboli during set up of the ablation unit. Optionally, the trap remains deployed during ablation and/or after ablation finishes and/or while the ablation unit is radially contracted. For example the embolic trap may prevent transport of emboli released when the ablation unit is contracted and/or peeled away from the walls of the lumen. Optionally the order of deployment may be fixed. For example extending a handle on the proximal end of a shaft may first spread the embolic trap at the distal end of the tool and then spread the ablation unit located proximal to the embolic trap. For example retracting the handle may first contract the embolic trap and then contract the ablation unit.

Alternatively or additionally, spreading of the ablation unit and the embolic trap may be by separate mechanisms and/or the operator of the device may control spreading of each unit independently.

An aspect of some embodiments of the current invention relates to an in-lumen dispersive electrode mounted on a shaft of an ablation unit. Optionally the ablation unit includes multiple pairs of ablation electrodes. The dispersive electrode may be located at a fixed distance of for example between 5 to 50 mm from the ablative electrodes. The dispersive electrode may be for example between 3 to 20 times as long as each ablation electrode. The dispersive electrode may serve as a return electrode for unipolar ablation.

Optionally, the dispersive electrode and/or the ablation electrodes are located in a geometry that makes it easy to recognize the location and/or orientation of the tool, for example using fluoroscopy. For example, the ablation electrodes may be arranged in a pattern near the distal end of the catheter and/or the dispersive electrode may be located on the shaft proximal to the ablation electrodes. For example the dispersive electrode may be located in a region between 2 mm and 300 mm from the ablation electrodes and/or between 5 mm to 200 mm from the ablation electrodes and/or between 5 mm to 100 mm from the ablation electrodes.

The dispersive electrode may be mounted on the same shaft as an ablation unit. The dispersive electrode and ablation unit are optionally inserted together into a lumen, for example from a single channel of a catheter. Optionally, the dispersive electrode and the ablation unit fit together into a single channel of a catheter. The electrodes may be configured to operate in unipolar and/or bipolar modes.

An aspect of some embodiments of the current invention relates to a method of catheter ablation wherein ablation progress may be measured locally at the site of one, some and/or all ablation electrodes. For example, during a pause in a bipolar ablation signal, ablation progress may be measured locally at an ablation electrode. For example local measuring of ablation progress may include measuring impedance, slew rate and/or propagation time of an auxiliary signal between the ablation electrode and a dispersive electrode. Alternatively or additionally, the impedance, slew rate and/or propagation time may be measured between a pair of ablation electrodes. Optionally when not ablating, an auxiliary signal may include an auxiliary current not meant to cause significant physiological effect. In some embodiments, measurements of an auxiliary signal may be made before ablation. The measurements may be used to determine a baseline behavior and/or to determine a location from which to apply an ablation signal.

An aspect of some embodiments of the current invention relates to a method of minimally invasive non-implantive neuromodulation for the treatment of neuro-immune disorders such as rheumatoid arthritis, inflammatory bowel disease, Crohn's Disease, myasthenia gravis, psoriasis, and/or inflammation-mediated diabetes, heart disease, and/or multiple sclerosis. Neuromodulation may be accomplished for example by ablation of splenic nerves and/or a carotid nerve (for example a carotid body).

In some embodiments nerves that signal the spleen may be modulated through local ablation of the splenic nerve. partial denervation may accomplish for example alleviation of rheumatoid arthritis [for example as documented by Boyle et al 2010] and myasthenia gravis [for example as documented by Bakhiet et al, 2006], as well as other inflammatory bowel diseases such as myasthenia gravis, psoriasis, diabetes, heart disease, and multiple sclerosis. Optionally, in accordance with some embodiments of the current invention, denervation may be accomplished by way of specialized catheters and apparatuses. For example, therapy may include the delivery of radio frequency (RF), microwave, ultrasound energy, injection of neurotoxic agents, the use of locally-applied heat and/or extreme cold. Therapy may be applied from within the splenic artery to partially destroy the sympathetic nerves that reach the spleen. For example therapy may be accomplished using a tool inserted into the splenic artery (for example by means of a catheter).

In some embodiments, carotid ablation may be achieved using a branched ablation catheter. For example, an ablation catheter may have an extendible/retractable member (for example a branch) that bifurcates away from the main catheter's body (the stem). RF energy may optionally be delivered between electrodes located on the stem and those located on the branch and/or between two branches. For example, a first branch may be located within the internal carotid artery and a second branch may be located within the external carotid artery. Optionally the path of RF currents is optimized to concentrate energy on the carotid body. For example, this may be further enhanced by cycling the delivery of currents between pairs of electrodes on the first branch and the second branch such that the delivery of energy is concentrated on the carotid body (which may be located at an intersecting region between the branches). Alternatively or additionally ablation of a carotid body may be achieved using an ablation catheter with a basket holding multiple electrodes and/or an insulating member. A catheter for carotid ablation may include an embolic trap and/or another protection member to remove emboli from a lumen and/or block transport of emboli along the lumen away from a treatment site.

An aspect of some embodiments of the current invention relates to a branching catheter including multiple branches bifurcating from a single stem. Each branch may include one or more electrodes from performing measurements of electrical properties and/or ablation of tissue. Individual branches may be steered into a lumen and/or secondary branches of the lumen. For example, when an object to be ablated is located between two branches of an artery, a first branch of a catheter may be inserted into one of the two branches of the artery and a second branch of the catheter may be inserted into the other branch of the artery. An electrical signal (for example a RF signal) may be passed from an electrode on one branch of the catheter through the object to an electrode located on the other branch. Alternatively or additionally, signals (for ablation and/or measurement) may be transported between electrodes on a single branch. For example, ablation may be performed simultaneously in multiple locations.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Embodiments

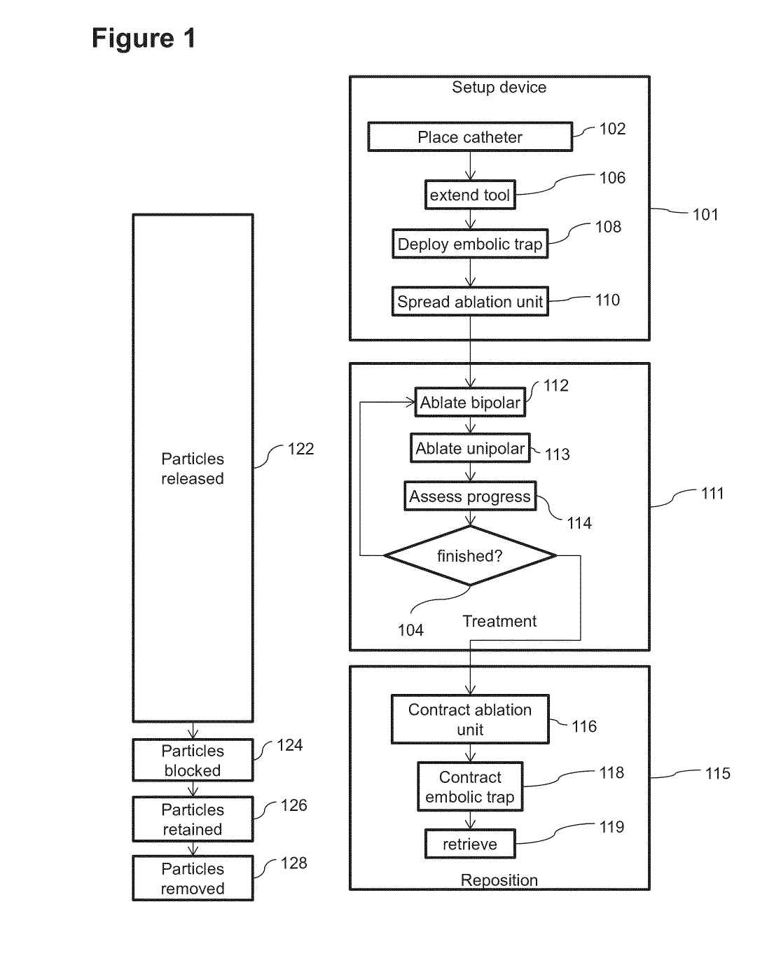

FIG. 1 is a flow chart illustration of a method of radio frequency ablation including embolic protection in accordance with an embodiment of the current invention. During ablation and/or after ablation when an insulator is being contracted emboli (particles) may escape into the lumen. Optionally, these particles will be trapped by the embolic protection trap. For example the trap may remain in place while the electrodes and/or an insulator (for example blood-exclusion membrane) has started to peel away from the vessel's wall. Optionally, clots and other debris caused by ablation may be safely retained in the trap while the catheter is removed from the body. An operator may control deployment, retrieval, movement and/or redeployment of the tools from a proximal end of the catheter outside of a patient.

In some embodiments, a device may be setup 101 in a treatment location. For example the device may include a catheter containing a tool (for example a catheter may include a guidewire, a guidewire channel and/or a sleeve). A distal end of the catheter may be placed 102 in a lumen near a treatment site. A tool may be extended 106 out of a distal opening of the catheter. For example the tool may include one or more units, for example a dispersive electrode and/or one or more pairs of ablation electrodes and/or an insulator (for example a blood exclusion membrane).

Alternatively or additionally, a dispersive electrode may be located on the outside of the catheter. Optionally the units may all be extended together (for example units may be located at fixed locations along the longitudinal axis of the tool and they may be extended together out of the catheter). Alternatively or additionally, there may be a separate control for one or more units which may be extended 106 separately.

In some embodiments an embolic trap may be deployed 108. For example deploying the trap may include spreading a cup shaped filter (for example a net and/or a porous membrane mounted on a frame) to cover the cross section of the lumen and/or to contact the inner walls of the lumen. Optionally the trap when deployed may block movement of particles inside a lumen. The embolic trap when deployed 108 may optionally allow fluid flow in the lumen.

In some embodiments, ablation electrodes and/or an insulator may be spread 110. For example, the after deploying 108 the embolic trap, the electrodes and/or the insulator may be spread 110 in a predetermined pattern along the walls of the lumen.

Optionally, deployment 108 of an embolic trap and/or spreading 110 of the ablation unit may be in a fixed order. Additionally or alternatively, the order and/or timing of deployment 108 of an embolic trap and/or spreading 110 of the ablation unit may be separately controlled by an operator.

In some embodiments after setting up 101 the tool, a treatment 111 may be performed. For example treatment may include bipolar ablation 112, unipolar ablation 113 and/or assessing progress of ablation 114.

In some embodiments, after ablation, the tool may be repositioned 115. For example repositioning may include radially contracting 116 the ablation unit and/or away from the walls of the lumen and/or folding 118 (for example collapsing and/or contracting) the embolic trap and/or the retrieving the trap and/or the ablation tool into the catheter 119. Alternatively or additionally, repositioning may include removing the tool from the patient and/or moving the tool within the patient to perform a further treatment in another location.

In some instances, embolic particles may be formed and/or released 122. For example particles may be released during spreading 110 of the ablation unit, during the treatment 111 and/or during contraction of the ablation unit away from the walls of the lumen. Optionally, the embolic trap will block 124 particles from being swept along with the blood to other parts of the body. Optionally, the embolic particles are retained 126 on the embolic trap. When the trap is folded 118 the embolic particles may be retained 126 for example in the folds of the trap and/or by adsorption and/or adhesion to the trap. Optionally when the trap is returned 118 out of the patient, the particles are also removed 128 with the trap.

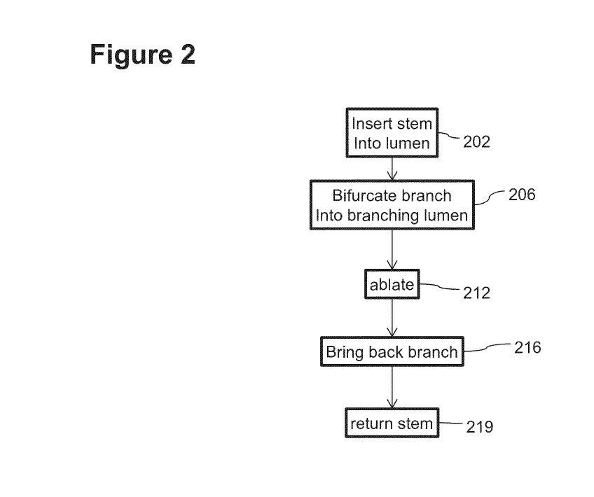

FIG. 2 is a flow chart illustration of a method ablating a tissue in a patient using a branching catheter in accordance with an embodiment of the current invention.

In some embodiments, when a body to be ablated is located near a junction of two lumens, a stem of a branching catheter may be inserted 202 into one of the two lumens. One or more branches of the catheter may be bifurcated 206 into the other of the two lumens. The body may be ablated 212, for example, by transmitting a radio frequency signal between an electrode on the branch and an electrode on the stem and/or between electrodes located on different branches. For example, a stem of a catheter may be inserted 202 into an internal carotid artery and/or a branch may bifurcate 206 into an external carotid artery (or vice versa) and/or a radio frequency signal may be passed between an electrode on the branch and an electrode on the stem to ablate 212 a carotid body.

In some embodiments, a branched catheter may be used to ablate 212 structures along the wall of one or more branching lumens. For example a radio frequency signal may be transmitted between two electrodes on the stem of the catheter to ablate structures in a first lumen. Alternatively or additionally, a radio frequency signal may be transmitted between two electrodes on a branch of the catheter to ablate structures in a branching lumen. Alternatively or additionally a catheter may have multiple branches and signals may be transmitted between branches. Optionally, signals may be transmitted simultaneously between multiple pairs of electrodes, speeding up the ablation of a large number of regions.

In some embodiments, a branching catheter may be used for exploratory and/or diagnostic procedures. For example, rather than transmitting an ablation signal between the electrodes, an exploratory signal may be transmitted (between two branches, between a branch and the stem, between two electrodes on the stem and/or between two electrodes on a single branch). The state of a structure may be inferred from a measure of the transmission of an exploratory signal and/or an of an ablation signal. For example, certain values and/or changes in impedance, slew rate and/or propagation time may signal the presence of a structure and/or a progress of an ablation.

In some embodiments interactions between branches of a catheter may be used to relocate the branches, move tissue and/or measure tissue properties (for example pliability). For example, a magnetic signal may be transmitted between two branches and/or between a branch and a stem. The magnetic signal may be used to pull two electrodes closer to each other, to push two electrodes apart, to measure the relatively locations of two branches and/or to measure the hardness of tissue between the magnets and/or squeeze tissue between the magnets.

Optionally, at the end of the procedure, the branch may be retracted back 216 to the stem and/or the stem (and/or the branch and/or the entire catheter and/or an associated tool) may be returned 219 back out of the patient.

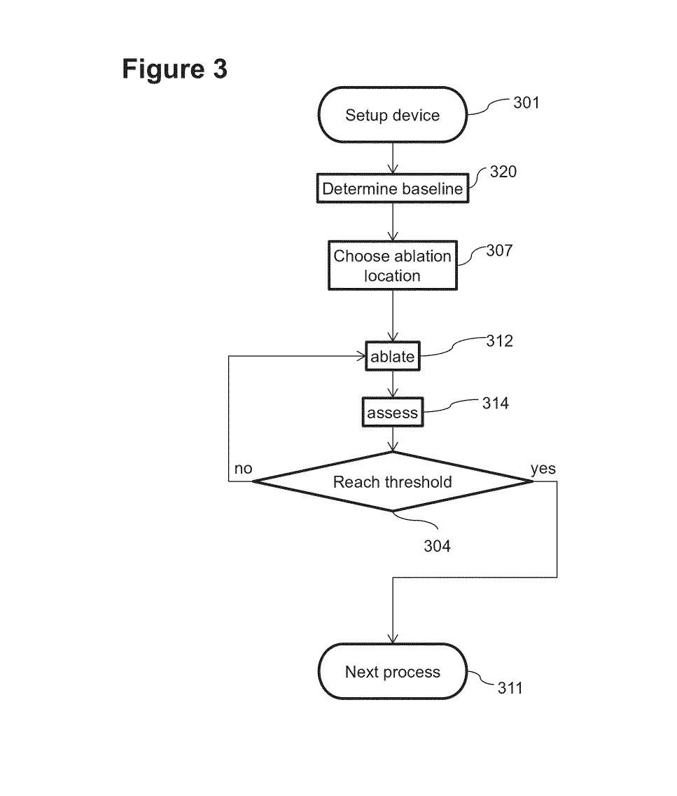

FIG. 3 is a flowchart illustration of a method of assessing ablation progress.

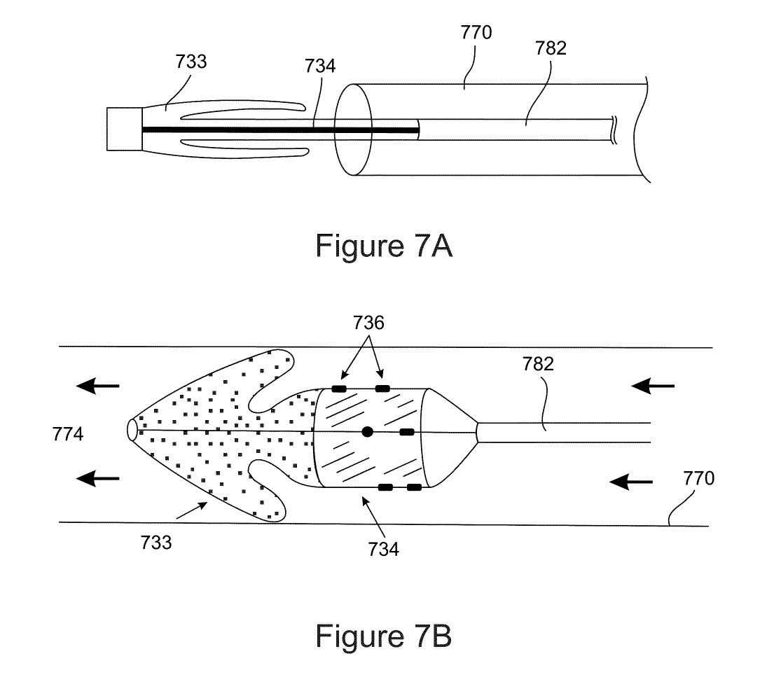

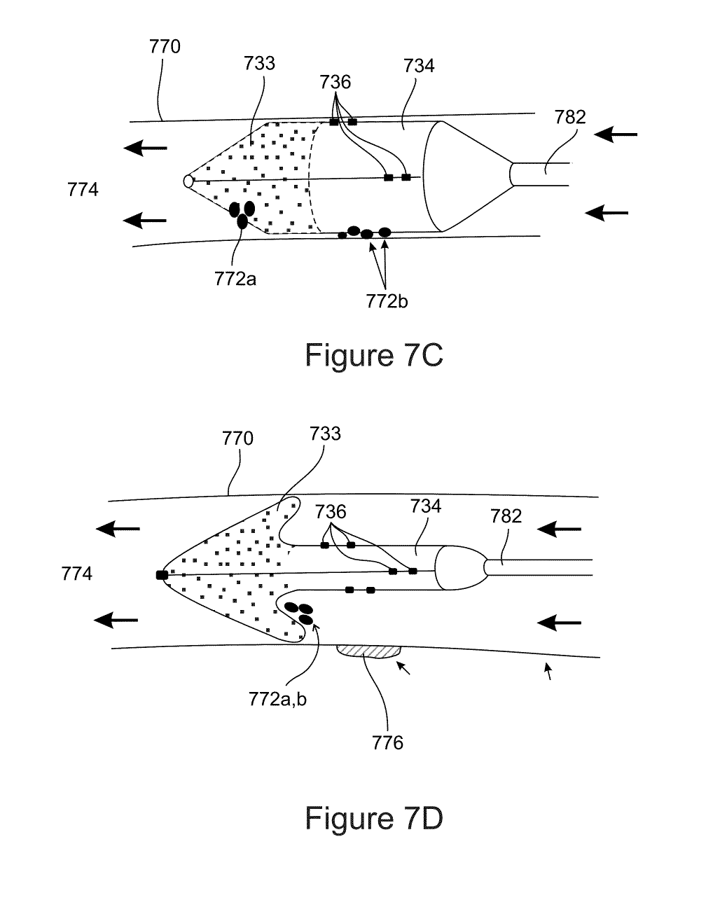

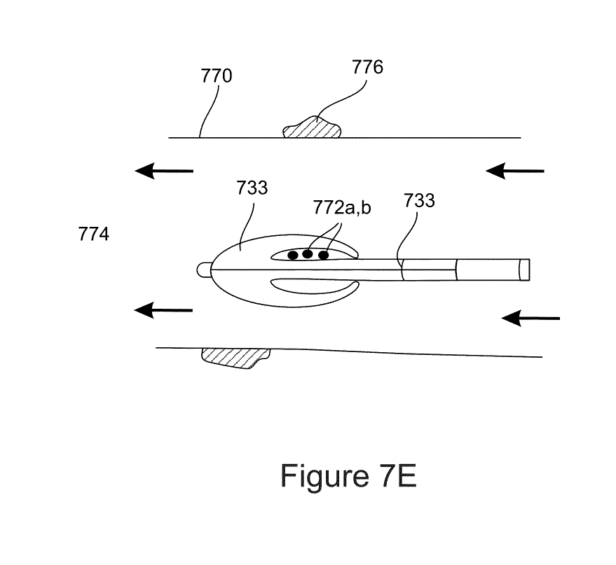

Optionally a device will be set up 301 for example by setting out electrodes in contact with tissue to be treated. Optionally the electrodes will be set out in a predetermined configuration (for example as described in FIG. 1 set up 101 and/or as illustrated for example in FIG. 7C).

In some embodiments before ablating tissue baseline behavior of the tissue will be determined 320. For example, test signals may be transmitted through the tissue between pairs of ablation electrodes and/or between electrodes of different pairs and/or between an ablation electrode and a disperse electrode. The impedance, slew rate and/or propagation time of signals may be measured between various electrodes.

Optionally a test signal will include a low current signal that does not damage the tissue.

In some embodiments, based on predetermined geometric criterion and/or based on the results of the baseline determination 320 sites and/or electrodes will be chosen 307 for Ablation. In some embodiments, ablation 312 will be performed for example by applying a high current radiofrequency signal to the tissue. During ablation 312 impedance may optionally be measured as an indication of ablation progress.

In some embodiments, ablation progress will periodically be assessed 314. For example, ablation 312 may temporarily suspended and a set of test signals transmitted through the tissue. The behavior of the signals (for example impedance, slew rate and/or propagation time) will optionally be measured. Changes are optionally interpreted to deduce the progress of ablation. When changes pass a threshold 304, ablation is stopped and/or another process started 311. When the changes have not reached the threshold 304 the ablation 312 may be continued.

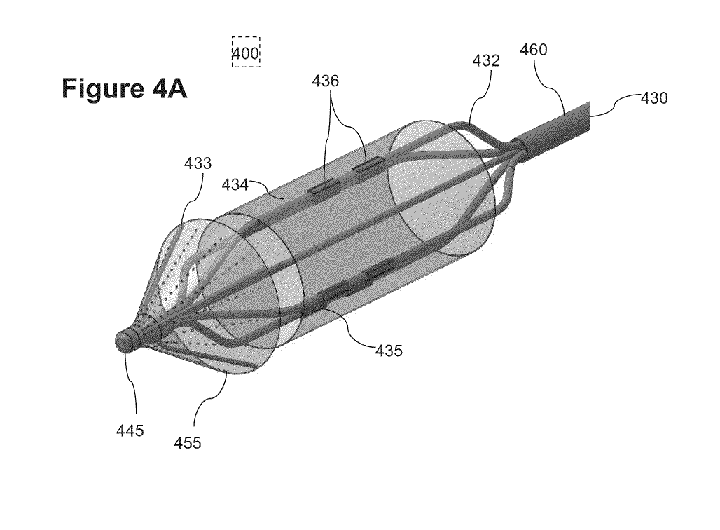

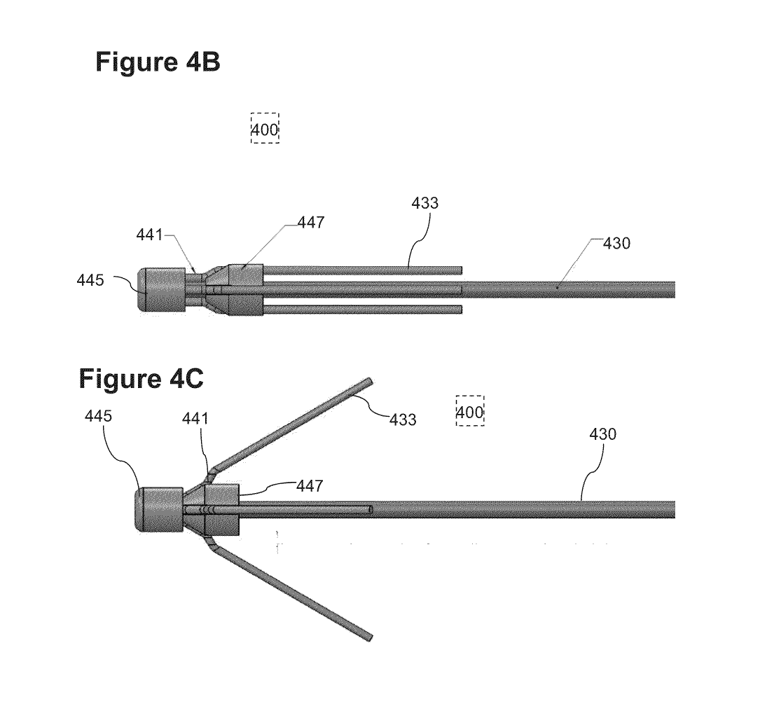

FIGS. 4A-C are perspective views of a tool 400 including ablation electrodes and an embolic trap on separate radially spreading frames attached to a single shaft in accordance with an embodiment of the current invention. Optionally a proximally located support structure spreads to hold an insulating membrane and/or ablation electrodes while a distally located support structure spreads to deploy an embolic trap. Ablation electrodes may also include sensors. For example ablation electrode sensors may be used to detect impedance, slew rate and/or propagation time.

FIG. 4A illustrates an embodiment of ablation tool 400 with an embolic trap in a fully deployed configuration. In the fully deployed configuration, tool 400 optionally includes the proximal support structure with supports 432, an insulator 434, and/or ablation electrodes 436 in a spread arrangement. Optionally, the proximally located radially spreading support structure includes a “basket” made for example out of nitinol wire spines and/or supports 432. Ablation electrodes 436 are optionally positioned on supports 432. Pairs of ablation electrodes 436 may be distributed along the periphery of the basket. Each support 432 may include one or more electrodes 436. Electrodes 436 may optionally be arranged in pairs. Pairs of electrodes 436 are optionally be staggered along the length of the basket (between the proximal end of the basket and the apex of the embolic trap located distal to the basket). In some embodiments, insulator 434 may include a polyurethane membrane. The membrane may be attached to the supports 432. The basket including supports 432 and/or insulator 434 may optionally radially contract to fit into a sheath 460 which fits into channel of a catheter. Optionally, the when tool 400 is extended out of the channel, the basket may be spread. In some embodiments, when the basket is spread, ablation electrodes 436 may optionally be arranged in contact with target tissue on the inner walls of a lumen in a patient. Optionally, some areas of electrodes 436 may be coated with an insulating coating 435. For example coating 435 may prevent shunting of current through lumen fluid. For example coating 435 may focus current to the area that is to be treated.

In some embodiments, an embolic trap may include struts 433 that are controlled separately from supports 432. Struts 433 are optionally located toward the distal end of tool 400 and/or distal of supports 432. In FIG. 4A struts 433 are spread radially to hold out a porous embolic protection membrane 455 like an umbrella. In the radially spread configuration, membrane 455 blocks a lumen of a patient. Pores are optionally large enough to allow fluid to pass along the lumen. The pores are optionally small enough to prevent embolic particles from traveling along the lumen past membrane 455. The ablation unit is optionally placed in the lumen so that flow in the lumen transports particles from the proximal end of tool 400 towards the distal end where the particles are trapped by membrane 455. For example, pore sizes may range between 30 and 150 μm and/or between 70 and 120 μm.

FIGS. 4B and 4C illustrate struts 433 of an embolic trap in a closed and open configuration respectively in accordance with an embodiment of the current invention. Optionally flexible shaft 430 includes an inner and an outer member.

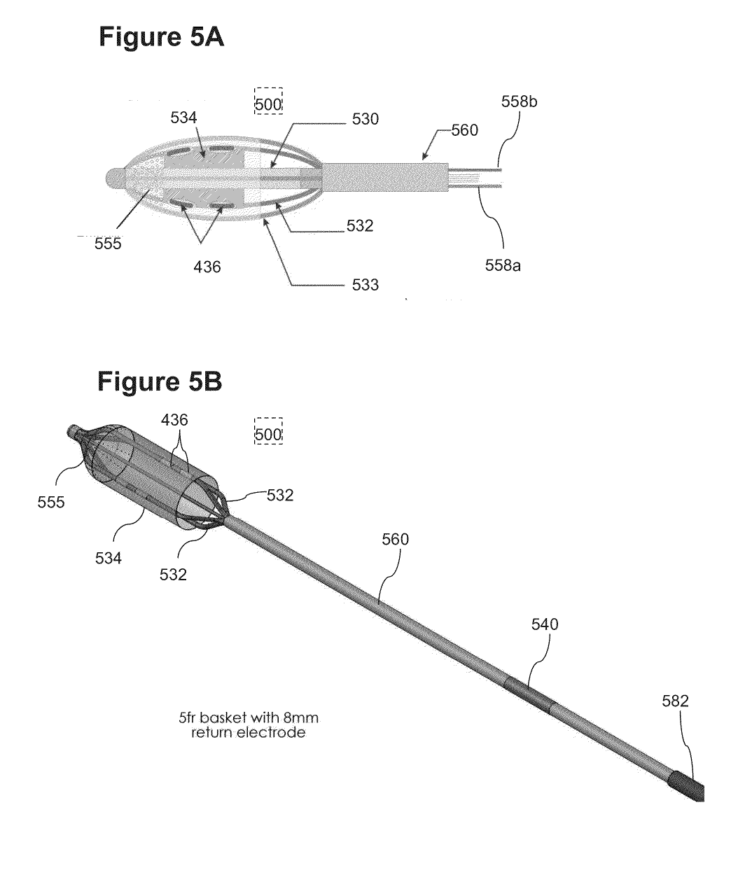

Optionally an embolic trap located near the distal end of the shaft is opened by pulling the inner member proximally with respect to the outer member. In some embodiments, shaft 430 may include a channel for a guidewire. In some embodiments a dispersive electrode (for example as shown in FIG. 5B) and/or an ablation basket (including for example supports 432, electrodes 436, and/or insulator membrane 434, for example as illustrated in FIG. 4A) may be mounted to the outer member.

Optionally the dispersive electrode and/or the ablation basket may be in a fixed longitudinal relationship to the embolic trap. Optionally and end cap 445 is mounted on the inner member.

In some embodiments, the embolic trap will have a cup shape (for example a conical cup for example as illustrated in embodiment 400 and/or a cylindrical cup and/or a rounded cup shape (similar to a bowl)). The cup may spread around an apex located along the axis of the basket supporting electrodes 436. The apex may be located distal to the basket (for example end cap 445).

FIG. 4B illustrates struts 433 in a closed configuration in accordance with an embodiment of the current invention. In the closed configuration the entire embolic trap may fit into the lumen of a catheter (for example a catheter may have an outer diameter of between 2 and 7 Fr.). Optionally, in the closed position an end cap 445 is displaced distally with respect to expansion struts 441 and an expansion wedge 447.

FIG. 4B illustrates struts 433 in an open configuration in accordance with an embodiment of the current invention. For example to open struts 433 an operator at the proximal end of a catheter pulls the inner member of shaft 430 proximally drawing end cap 445 towards wedge 447. In turn, end cap 445 may, for example, push expansion struts 441 onto wedge 447 forcing expansion struts 441 and struts 433 outward opening the embolic protection trap for example as shown in FIG. 4A.

FIGS. 5 A-B and 6 illustrate an ablation tool 500 with an integrated ablation unit and embolic trap in accordance with an embodiment of the current invention. For example embolic protection includes a porous membrane 555 attached to the distal end of a basket. Electrodes for radio frequency ablation are optionally attached to the basket proximal to porous membrane 555. Optionally an insulating membrane 534 is also attached to the basket proximal to porous membrane 555. Optionally porous membrane 555 and insulating membrane 534 may be made of a single sheet of material (for example polyurethane) with pores in the distal end. Alternatively or additionally, porous membrane 555 may be a separate from insulating membrane 534. For example porous membrane may be made of fibers and/or a porous polymer.

FIG. 5A illustrates the basket of tool 500 in accordance with an embodiment of the current invention. For example, an outer set of struts 533 carry embolic protection filter membrane 555, while an inner set of supports 532 carries ablation electrodes 536 and/or blood-exclusion insulating membrane 534. Optionally, radial expansion and/or radial contraction of outer set of struts 533 is controlled by a first puller wire 558a and/or radial expansion and/or radial contraction of inner set of supports 532 is controlled by a second puller wire 558b. Alternatively or additionally, a single puller wire may control both sets of supports 532 and struts 533. For example pulling the single wire a small distance would open struts 533 and the embolic trap and further pulling would open supports 532 along with electrodes 436 and/or membrane 534.

Optionally tool 400 is mounted on a shaft 530. When the basket of tool 400 is folded, the struts 533 and the supports may be arranged parallel to and closely packed around the axis of the basket. In the folded configuration, the entire assembly may fit into a sheath 560 which may fit into a channel of a catheter.

FIG. 5B illustrates tool 500 and a dispersive electrode 540 extended out of a 5 French catheter 582. Optionally the dispersive electrode 540 is larger than the ablation electrodes 436.

In some embodiments, a control unit may supply power for ablation (for example: a radio frequency (RF) generator). For example the control unit may be a rechargeable and/or battery-powered. The ablation generator may operate for example around the 460 kHz frequency and/or ranging for example between 400 and 600 kHz or other RF frequency ranges assigned to ISM (Industrial, Scientific, and Medical) applications within the low-frequency (LF: 30 to 300 kHz), medium-frequency (300 kHz to 3 MHz), and high-frequency (HF 3 to 30 MHz) portions of the RF spectrum. The control unit may have a number of channels that allow ablation to be conducted bipolarly between electrode pairs through the target tissue. The generator may optionally be able to deliver ablation energy to be conveyed simultaneously between one, some and/or all bipolar ablation electrode pairs in the catheter. For example a catheter may include four or more bipolar ablation electrode pairs. In some embodiments, the generator may supply a maximum power of, for example, between 3-10 W per bipolar channel. The generator may optionally be able to ablate unipolarly between one, some and/or all of the contact electrodes and a dispersive electrode, e.g., catheter-borne reference in-lumen dispersive electrode. Lesion formation may for example take between 15 to 180 seconds. Each channel may have a minimum voltage compliance of 100 V. In some embodiments, the minimum voltage compliance may permit, for example, an average of between 2 and 10 W to be delivered per bipolar electrode pair presenting an impedance for example ranging between 1.0 and 1.5 kΩ.

In some embodiments, an ablation electrode of the current invention may be made for example of between 80% and 95% Platinum and/or between 20% and 5% Iridium. The ablation electrodes may range for example between 0.5 and 4 mm long and/or have an electrically active area for example of between 0.1 and 1 mm² and/or have a diameter ranging from 0.01 to 0.05 inch (0.25 to 1.27 mm). The electrically active area of the ablation electrodes may be in contact with a target tissue. The distance between ablation electrodes may range for example between 0.5 and 3 mm or more.

In some embodiments, a dispersive electrode may for example have a length ranging for example between 4 to 20 mm and/or have a diameter ranging between 2 and 5 French (between 0.67 and 1.67 mm). The dispersive electrode may have an electrically active area ranging for example, 20 to 50 times or more than the electrically active area and/or surface of contact of the ablation electrodes. For example the electrically active area of the dispersive electrode may range between 50 to 150 mm² (e.g., between 50 to 100 mm2, between 100 to 150 mm2, between 75 to 120 mm2 etc.). Optionally the electrically active surface of the disperse electrode may be in electrical contact with a fluid in a lumen of a patient. In some embodiments, the dispersive electrode may be coated with a material such as porous titanium nitride (TiN) or iridium oxide (IrOx). The coating may increase microscopic surface area of the electrode in electrical contact with lumen fluid.

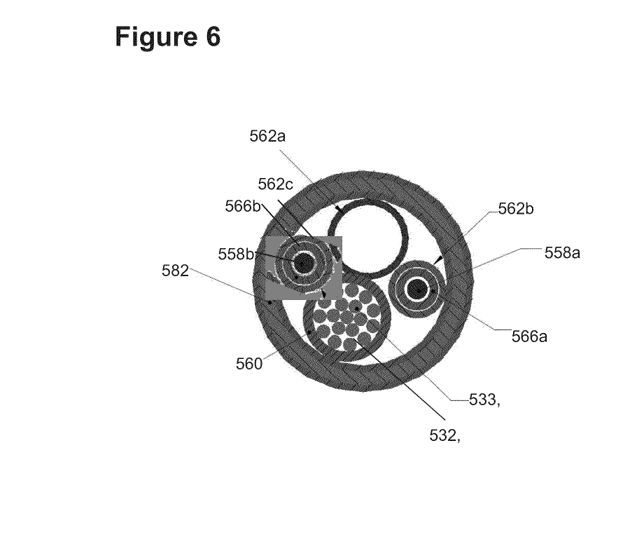

FIG. 6 illustrates a cross section of catheter 582 containing an ablation tool 500 in accordance with an embodiment of the current invention. The inner diameter of catheter 582 may for example range between 1.2 to 1.28 mm. Outer sheath 560 (which may be made for example of Teflon) may contain struts 533 and/or supports 532 which may each be made for example of wire between 40 to 45 gauge (for example 0.07 to 0.12 mm diameter nitinol wire and/or flat nitinol wire). The catheter optionally includes a first guidewire channel 562a and/or one or more pullwire channels 562b, 562c. A first pullwire channel 562b may contain a pull wire 558a and/or a compression coil 566a. A second pullwire channel 562c may contain a pull wire 558b and/or a compression coil 566b.

FIGS. 7A-D show an ablation tool with embolic protection at four stages of deployment in accordance with an embodiment of the current invention. When completely contracted, the tool optionally fits within a catheter 782. Catheter 782 may be inserted into a lumen 770 of a patient (for example a splenetic artery). Optionally, after the tool is extended out of the catheter, an embolic trap 733 is deployed to block embolic particles from traveling away from the treatment site. Further expansion optionally spreads and arranges the ablation unit (for example placing ablation electrodes against a wall of the lumen). Optionally, the embolic trap remains in place during treatment and/or until the ablation unit is contracted. Finally, the embolic trap may be folded and/or the emboli may be trapped and/or retrieved with the trap into the catheter and/or returned out of the patient.

FIG. 7A shows a tool being extended out of a catheter in a folded configuration in accordance with an embodiment of the current invention.

FIG. 7B shows a tool at the beginning of expansion in accordance with an embodiment of the current invention. As the device is radially spread, the embolic trap 733 is optionally deployed in contact with the walls of lumen 770 before the electrodes 736 and/or insulator 734 are arranged for treatment. Fluid may optionally continue to flow 774 through lumen 770 through pores in embolic trap 733. Particle larger than the pores of membrane (for example particles larger than 0.05 mm and or particles larger than 0.1 mm) are optionally blocked by embolic trap 733.

FIG. 7C shows a tool in a fully expanded state in accordance with an embodiment of the current invention. In the fully expanded state insulator 734 may inhibit shunting of electrical current from electrodes 736 through fluid flowing 774 in lumen 770. In some embodiments, fluid flowing 774 along the inner surface of insulator 734 may cool the ablation zone and/or electrodes 736. In a case where the treatment produces particles 772a, the particles may be released and trapped immediately by embolic trap 733. Alternatively or additionally, some embolic particles 772b may be trapped on and/or between insulator 734 and/or electrodes 736 and/or the walls of lumen 770.

FIG. 7D shows a tool being radially contracted after treatment in accordance with an embodiment of the current invention. As the insulator 734 is radially contracted, the electrodes and/or insulator 734 will optionally disengage from the wall of lumen 770 before the embolic trap 733 is folded. As shown for example in FIG. 7D, particles 772b (for example blood clots other debris) formed at an ablation site 776 may dislodge. Flow 774 may bring particles 772b to embolic trap 733 where they will optionally be trapped by the embolic trap 733.

FIG. 7E shows a tool as embolic protection trap 733 is folded for retrieval to the channel of the catheter in accordance with an embodiment of the current invention. Optionally, trap 733 folds over particles that were blocked by the embolic protection trap 733. As the catheter and/or tool is removed from the body particles 772a,b are also optionally removed.

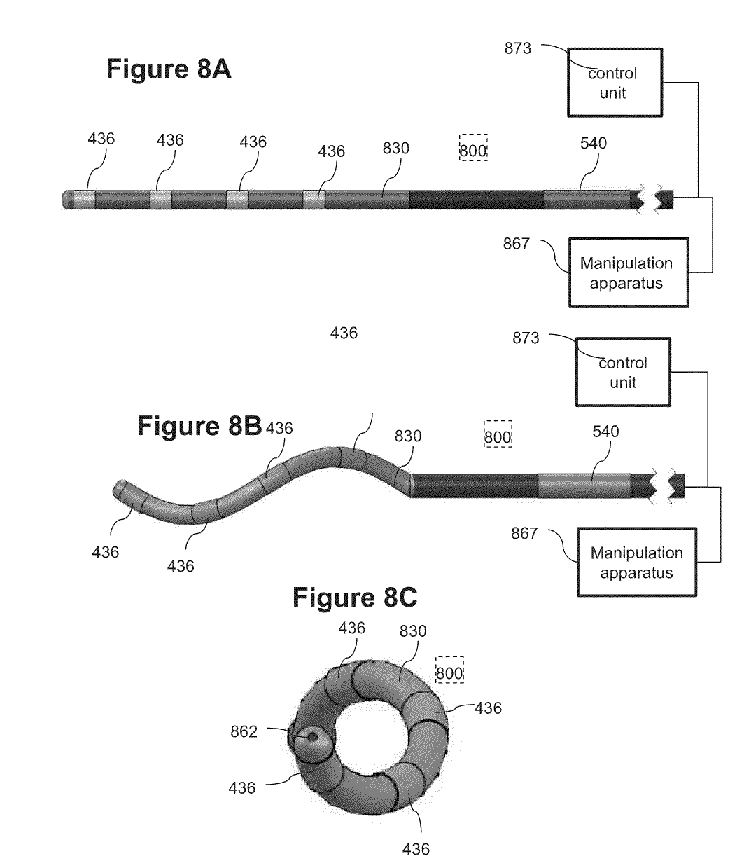

FIGS. 8A-C illustrates a single shaft ablation device 800 in accordance with an embodiment of the current invention. Optionally, the single shaft includes a plurality of ablation electrodes. The electrodes may be spread radially by bending the shaft into a helical structure. The helical structure has a lateral diameter which is adapted to the size and shape of a lumen for example of a blood vessel. Optionally when the shaft is bending the shaft to the helical configuration brings ablation electrodes into contact with the lumen walls.

Optionally device 800 may include multiple electrodes on a single shaft. The shaft optionally has a first configuration wherein the shaft may be straight and/or very thin and/or supple for insertion into very thin lumens and/or a lumen that has very sharp turns. An operator standing outside the lumen may switch the device, for example using a manipulation apparatus 867, from the first configuration to a second, radially spread configuration. For example in the radially spread configuration, the shaft bends to form a three dimensional helix that is circumscribed by and contacts the inner wall of the lumen at various points around the circumference of the lumen thereby pushing the electrodes against the walls of the lumen.

FIG. 8A illustrates device 800 in a first straight and/or longitudinally stretched configuration in accordance with an embodiment of the current invention. In the straight configuration shaft 830 may have a diameter ranging for example between 0.2 and 2 mm. Device 800 may include for example a channel 862 for a guide wire and/or a pull wire. For example in the first configuration device 800 may be inserted into a lumen having a diameter of between 1 to 2 mm and/or a lumen of greater than 2 mm and/or a lumen of less than 1 mm. For example, device 800 in the first configuration may be inserted into a lumen having a radius of curvature of between 1 to 2 mm and/or between 1 to 5 mm and/or between 5 to 10 mm and/or greater than 10 mm.

FIGS. 8B and 8C illustrate longitudinal and axial views of device 800 in a radially spread configuration in accordance with an embodiment of the current invention. For example, an operator at the proximal end of a catheter causes device 800 to contract longitudinally and/or spread radially for example from the configuration of FIG. 8A to the configuration of FIG. 8B,C. The radial spreading will optionally push and/or arrange electrodes 436 against the walls of a lumen. For example, in FIGS. 8B,C the device has formed into a spiral and/or helix. The helix is optionally spread radially to contact the inner walls of the lumen around the circumference thereof.

In some embodiments, an operator may pull on a puller wire to cause device 800 to shorten in the longitudinal direction and/or spread radially and/or spiral.

Alternatively or additionally shaft 430 may include a nitinol component that changes shape due temperature changes. In some embodiments device 800 may include a control unit 873 for example to control signals transmitted by electrodes 436 and/or to measure for example impedance, slew rate and/or propagation time.

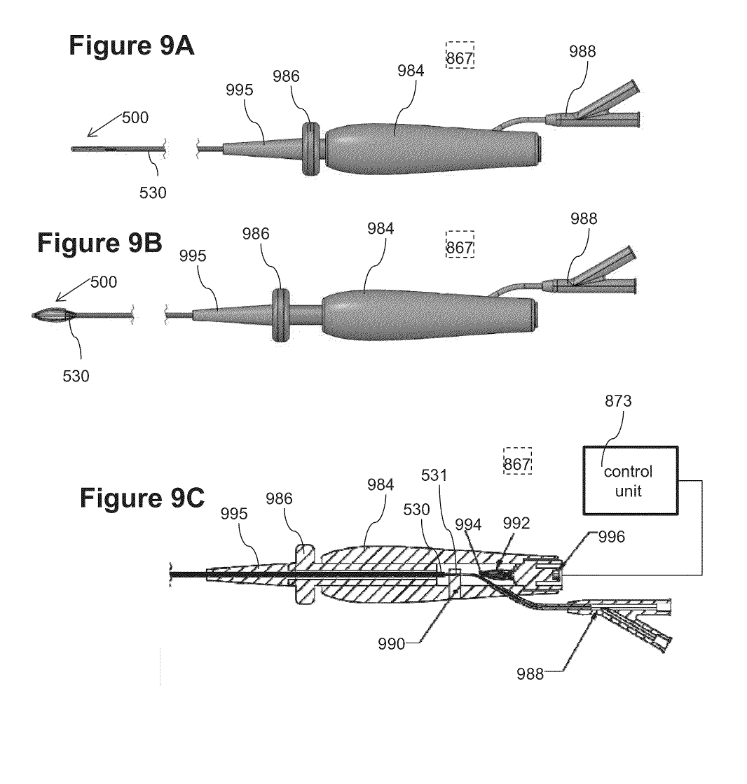

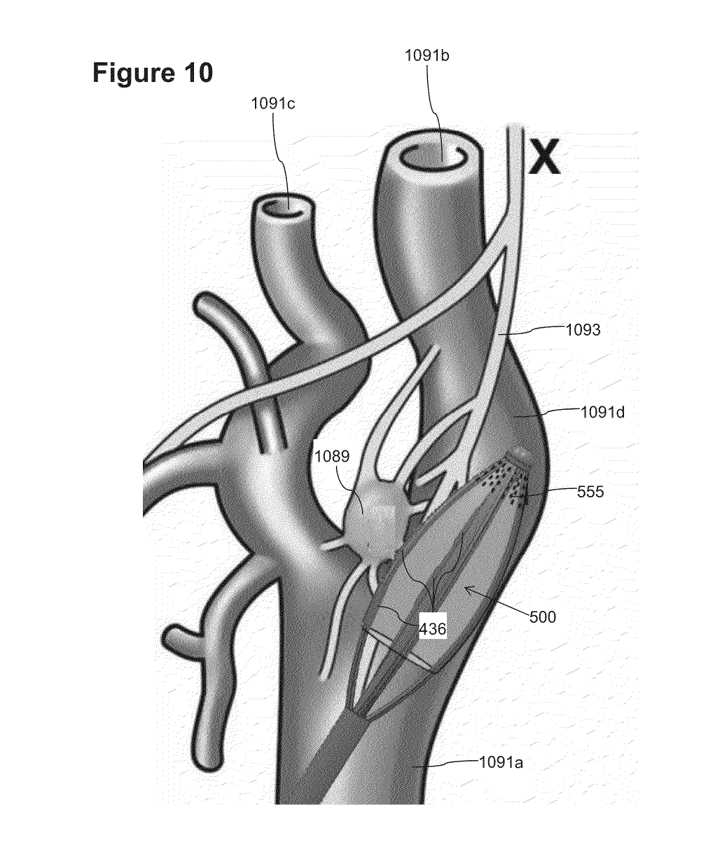

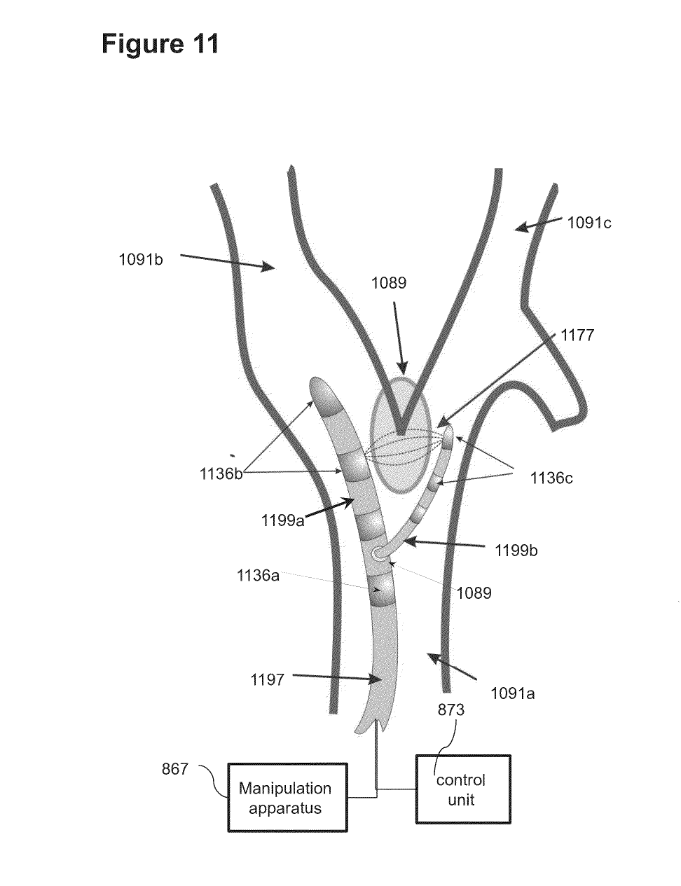

FIGS. 9A-C illustrate a manipulation apparatus 867 for an ablation tool in accordance with some embodiments of the current invention. A tool (for example ablation tool 500) is attached to the distal end of a shaft (for example shaft 530). Shaft 530 passes through a catheter (for example a 5 Fr. Catheter). A manipulation apparatus 867 is optionally attached to the proximal end of the catheter and/or shaft 530). Alternatively or additionally manipulation apparatus 867 may be used with spiraling catheter (for example as illustrated in FIGS. 8A-C) and/or a branching catheter (for example as illustrated in FIGS. 10-11).

FIG. 9A illustrated a manipulation apparatus 867 and tool 500 in a contracted state in accordance with some embodiments of the current invention. For example, when a control knob 986 is in a proximal position, the basket of tool 500 is contracted. In the contracted configuration, the basket that supports of the electrodes may be collapsed around its axis. For example supports of the basket are optionally arranged parallel to each other along the axis of the basket and/or axial to shaft 530.

Optionally, in the contracted state, tool 500 may fit into a channel of a catheter.

FIG. 9B illustrates a manipulation apparatus 867 and tool 500 in a radially expanded state in accordance with some embodiments of the current invention. For example, when a control knob 986 is in a distal position, the basket of tool 500 is radially spread. Alternatively or additionally when knob 986 is drawn back to a fully proximal position a tool may be in a fully contracted state (for example as illustrated in FIG. 7A) and/or when knob 986 is partially drawn back to an intermediate position a tool may be in an intermediate state (for example as illustrated in FIG. 7B wherein the embolic trap is deployed, but the ablation basket is contracted) and/or when knob 986 is pushed forward to a fully distal position a tool may be in a fully expanded state (for example as illustrated in FIG. 7C). Alternatively or additionally, for a spiraling catheter when knob 986 is in the proximal position the catheter may be in the first (straight) configuration (for example as in FIG. 8A) and/or when knob 986 is in the distal position the catheter may be in the second (radially expanded) state (for example as in FIGS. 8B-C). Alternatively or additionally, for a branching catheter when knob 986 is in the proximal position the may be retracted and/or when knob 986 is in the distal position the branch may be extended.

In some embodiments, the manipulation apparatus 867 optionally includes a luer adaptor 988 for example for insertion of a guidewire and/or fluid. The manipulation apparatus 867 optionally includes a handle 984 used by an operator for example for holding the apparatus and/or for extending the tool out of the distal end of the catheter and/or for retrieving the tool. The manipulation apparatus 867 optionally includes a strain relief bore 995 for example for directing the proximal end of a catheter.

FIG. 9C is a cross section illustration of a manipulation apparatus 867 in accordance with some embodiments of the current invention.

In some embodiments, the outer member of shaft 530 is connected to control knob 986 and/or an inner member 531 of shaft 530 is connected to an anchor point 990 in handle 984. Optionally, control knob 986 slides longitudinally with respect to handle 984. For example, when a control knob 986 is in a proximal position, the outer member of shaft 530 is pulled back with respect to inner member 531 radial contracting a basket of an ablation device 500 (for example by pushing an end cap away from the spines and/or supports allowing the supports to lie flat along the axis of the basket). For example, when control knob 986 is in a distal position, the outer member of shaft 530 is pushed forward with respect to inner member 531 opening a basket of an ablation device 500 (for example by pushing the proximal end of the spines and/or supports distally, sandwiching the spines and/or supports between and end cap and the outer shaft causing the supports to bulge radially away from the axis of the basket).

Lure adapter 988 may optionally be connected to a channel passing through the center of shaft 530 and/or to a channel in an outer catheter. A multi pin electrical connector 996 is optionally connected via lead wires 992 to electrodes, thermocouples and/or other electrical devices in tool 500. Tubes 994 may connect luer adapter 998 to various channels of the catheter. A control unit 873 may be connected to connector 996. Control unit 873 may detect signals and/or control signal generation using sensor and/or electrodes of the ablation tool. For example a control unit may detect temperature and/or slew rate of a signal and/or propagation time of a signal and/or impedance.

FIG. 10 illustrates use of a tool 500 for ablating a carotid body 1089 in accordance with an embodiment of the current invention. For example, a catheter is inserted through the common carotid artery 1091a to the junction between the internal carotid artery 1091b and the external carotid artery 1091c and/or to a carotid sinus 1091d. Optionally test signals may be used to determine which electrodes are located close to a target (for example a carotid body 1089 and/or a carotid sinus nerve 1093).

Optionally, ablation signals may be transmitted between one or more pairs of electrodes to ablate one or more targets. An embolic trap membrane 555 may protect the patient from emboli.

FIG. 11 illustrates use of a branching catheter to ablate a carotid body in accordance with an embodiment of the current invention. A branching catheter may include a stem with a junction. One or more branches may divide off from the stem at the junction. Each branch may include one or more electrodes. Optionally each branch of the catheter may be inserted in to a separate lumen at a junction between two lumens. An electoral signal may then be passed from an electrode on one branch to an electrode on the other branch, for example to ablate an object located near the junction between the two lumens.

In some embodiments a stem 1197 of the catheter is inserted into common carotid artery 1091a. Optionally a first branch 1199a of the catheter is inserted into inner carotid artery 1091b. A second branch 1099b may bifurcate from stem 1197 at a junction 1089. Optionally, the second branch is extended and/or retracted into and/or out from junction 1089. For example, an operator may control extension and/or contraction of second branch 1099b from a proximal end of the catheter using a manipulation apparatus 867. The second branch is inserted, for example, into an outer carotid artery 1091c. An ablation signal 1177 may be transmitted from an electrode 1136b on the first branch to an electrode 1136c on the second branch. Alternatively or additionally a signal may be transferred between a pairs of electrodes 1136b on the first branch 1199a and/or between a pairs of electrodes 1136c on the second branch 1199b and/or between a pairs of electrodes 1136a on the stem 1197. Optionally a pattern of signals may be transmitted to chosen electrodes to best ablate the tissue with minimum collateral damage.

In some embodiments, the distance between electrodes pairs used for transferring a signal between different branches of the catheter (for example between electrodes 1136c and electrodes 1136b may range between 10 and 60 mm and/or between 15 and 40 mm).

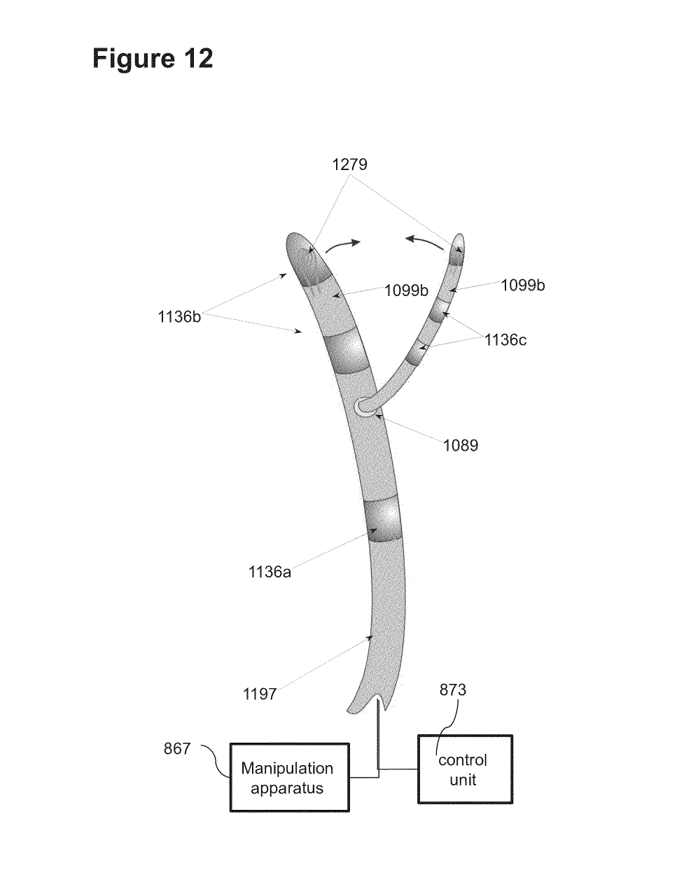

FIG. 12 illustrates a branching catheter in accordance with an embodiment of the current invention. A branching catheter may optionally include sensors and/or actuators to sense or create interaction between branches.

In some embodiments, permanent magnets and/or energizable electromagnets 1279 may cause attraction between the distal portions of a catheter's bifurcating branches 1099a,b. For example the magnets 1279 may be used to ensure proper relative location between the electrodes on opposing branches. The strength of the attraction may be controlled such that appropriate contact between the electrodes and the artery walls is accomplished.

Further Optional Features

In some embodiments, the ablation electrodes may be mounted on a support structure. For example a support structure may include a radially spreading frame.

Optionally the frame in the spread state may hold the electrodes against the walls of a lumen under treatment. For example the lumen may include a blood vessel with a diameter ranging between 1 and 4 mm and/or between 4 and 8 mm and/or between 8 and 20 mm. Optionally the electrodes may be held in a fixed pattern against the lumen walls. For example the electrodes may be arranged in pairs. The distance between electrodes of a pair of electrodes may range, for example between 1 and 6 mm. For example pairs of electrodes may be arranged around the lumen in a helical pattern. In the radially spread configuration, the distance between electrode pairs may range for example between 2 and 15 mm. For example the support structure and/or frame may include a radially spreading basket and/or a reconfigurable shaft. For example, a reconfigurable shaft may have a first configuration which is longitudinally stretched and/or flexible and/or straight. For example, a reconfigurable shaft may have a second configuration which is laterally spread. In the first configuration the shaft may fit and/or be transported along a narrow channel and/or lumen. For example in the laterally spread configuration the shaft may for a spiral and or a helix. In some embodiments, in the laterally spread configuration, the electrodes may be pushed up against the walls of a lumen.

In some embodiments, an ablation tool may include an insulator (for example an insulator may include a blood exclusions member). For example the support structure holding the electrodes may include a balloon and/or a membrane. The blood exclusion member may in some embodiments inhibit shunting of electrical signals through lumen fluids. Alternatively or additionally the blood exclusion member may prevent particles from the treatment sight from entering the blood and/or forming an embolism.

Some embodiments of the current invention may include a multi-electrode ablation tool. The device may be inserted into a body lumen via a catheter. At times the ablation tool may be referred to as an ablation catheter or a catheter. A multi-electrode ablation tool may be powered by a control unit. The control unit may include, for example, an RF generator. The control unit may have a number of channels that convey an electrical signal bipolarly through a target tissue between electrode pairs (for example, the ablation electrodes may be mounted on the catheter's working [distal] end), and/or unipolarly through a target tissue between an ablation electrode and a dispersive (reference) electrode (e.g., a shaft electrode in contact with lumen fluid (for example blood) and/or an external electrode). The electrodes may be activated in accordance with a switch configuration set by a multiplexer. Multiplexer RF channels may be used to transmit radio frequency (RF) ablation energy to the electrodes. The RF channels may optionally be used to transmit an auxiliary signal. For example an auxiliary signal may be used to measure impedance, slew rate and/or propagation time between pairs of electrodes. When measuring impedance, slew rate and/or propagation time a sensor may optionally include an electrode. In some embodiments a sensor for measuring impedance, slew rate and/or propagation time may include one or more of an ablation electrode and/or a dispersive electrode. For example an auxiliary signal may be similar to an ablation signal but at a lower power (optionally minimizing and/or avoiding tissue damage during measurements). The RF channels may optionally include means to measure electrode/tissue impedance, slew rate and/or propagation time. In some embodiments, measurements may be made with high accuracy and/or repeatability. The RF channels may optionally be controlled by a controller (e.g., a microcontroller and/or single-board computer). The channels may optionally be capable of generating stimulation signals to evoke a response from target tissues and/or measuring an evoked signal from the target tissue. For example, the control unit may transmit a nerve stimulating signal over an electrode (for example an electrode of the ablation catheter). For example, the control unit may evaluate an electrical signal transmitted by the target tissue and/or sensed by an electrode (for example an electrode of the ablation catheter).

Optionally a catheter according to some embodiments of the current invention may be used for renal, splenic and/or carotid denervation. Denervation, may include, for example, a minimally invasive, endovascular catheter based procedure using radiofrequency ablation aimed at treating resistant autoimmune disease and/or hypertension. Radiofrequency pulses may be applied to a renal artery, splenic artery and/or a carotid artery. Ablation in some embodiments may denude nerves in the vascular wall (adventitia layer) of nerve endings. This may causes reduction of renal sympathetic afferent and efferent activity and/or blood pressure can be decreased and/or autoimmune diseases may be mediated and/or swelling may be reduced. During the procedure, a steerable catheter with a radio frequency (RF) energy electrode tip may deliver RF energy to an artery for example via standard femoral artery and/or radial access and/or through the aorta. A series of ablations may be delivered along each artery.

As used herein, the term “controller” may include an electric circuit that performs a logic operation on input or inputs. For example, such a controller may include one or more integrated circuits, microchips, microcontrollers, microprocessors, all or part of a central processing unit (CPU), graphics processing unit (GPU), digital signal processors (DSP), field-programmable gate array (FPGA) or other circuit suitable for executing instructions or performing logic operations. The instructions executed by the controller may, for example, be pre-loaded into the controller or may be stored in a separate memory unit such as a RAM, a ROM, a hard disk, an optical disk, a magnetic medium, a flash memory, other permanent, fixed, or volatile memory, or any other mechanism capable of storing instructions for the controller. The controller may be customized for a particular use, or can be configured for general-purpose use and can perform different functions by executing different software.

The controller may optionally be able to calculate the temperature of some or all of the electrodes and/or near some or all of the electrodes. For example, temperature measurements may be sensed by means of the thermocouple attached to each electrode and the output of the means is forwarded to the controller for calculation. Interaction with the user (e.g., a physician performing the ablation procedure) may optionally be via a graphical user interface (GUI) presented on for example a touch screen or another display.

In some embodiments, electrode impedance, slew rate and/or propagation time measurements may be used to estimate contact (estimated contact) between electrode and tissue as surrogate for thermal contact between electrode interface and target tissue (for example a low impedance of a unipolar signal between an ablation electrode and a dispersive electrode may indicate good contact between the ablation electrode and the target tissue). In some embodiments, power being converted to heat at electrode/tissue interface may be estimated (estimated power) for example based on the estimated contact, applied power and/or electrode temperature. Together with the time of RF application to the tissue, the estimated contact and/or estimated power and/or electrode temperature may optionally be used to calculate energy transferred to target tissue and/or resulting target tissue temperature locally at individual ablation electrode locations. Optionally, the results may be reported in real-time. Optionally, based for example on the calculated cumulative energy transferred to target tissue, the duration of ablation may be controlled to achieve quality of lesion formation and/or avoid undesirable local over-ablation and/or overheating. Control algorithms may deem to have completed lesion formation successfully for example when the quality of lesion at each electrode location reaches a predetermined range.

Some embodiments of the current invention may combine a multi-electrode ablation tool with blood exclusion. In some embodiments, the distance from the proximal end of the insulator to the distal end (toward the catheter tip) of an in-catheter dispersive electrode may range for example between 10 to 75 mm (e.g., between 10 to 15 mm, between 10 to 25 mm, between 25 to 50 mm, between 50 to 75 mm etc.). For artery denervation, the distance between the dispersive electrode and the proximal end of the spreadable structure may range preferably between 20 to 50 mm (e.g., 20 mm, 30 mm, 40 mm, 50 mm etc.) to ensure that the dispersive electrode is within the aorta, and away from the desired ablation area within the renal artery.

Various embodiments of the current invention may be configured to fit for example in a 5 French (1.33 mm diameter) catheter with a lumen extending from the handle through the distal tip making it possible to insert it with the aid of a standard 0.014 inch (0.36 mm) guide wire. The flexibility of the assembly may optionally be compatible with applicable medical standards. A catheter (for example the various embodiments described below) may include a guidewire. For example, the guidewire may be inserted through a lumen of the catheter. Optionally, the guidewire may help position the catheter. The guidewire may optionally be able to extend past an orifice at the distal end of the catheter.

In some embodiments in the radially spread configuration the distance between the most proximal ablation electrode and the most distal ablation electrode may range for example between 5 and 20 mm and/or between 20 and 50 mm and/or between 50 and 100 mm. In some embodiments the radius of the basket may range for example between 2 and 4 mm and/or between 4 and 8 mm and/or between 8 and 20 mm.

In some embodiments an ablation catheter may be used for neuromodulation of splenic nerves for control of autoimmune disorders. The spleen may be importance in mediating autoimmune disorders. For example, the spleen may manufacture immune cells. In the spleen, the immune and nervous systems may interact. For example, some researchers have concluded that the vagus nerve carries nerve fibers that directly modulate the production of inflammatory factors by macrophages in the spleen [Rasouli 2011]. Some researchers [Buijs et al. 2008] claim that the autonomic output of the brain is involved in the adaptive immune response, allowing information from the brain to the spleen to be translated into the generation of antigen specific antibodies, elucidating a mechanism by which mood; sleep and stress affect the immune response of the body.

Studies on electrical stimulation of the vagus nerve have indicated that the body's inflammatory reflex can be artificially modulated to dampen inflammation and improve clinical symptoms of auto-inflammatory diseases such as rheumatoid arthritis and Crohn's Disease. Methods to treat these diseases may involve the use of a vagus-nerve stimulator that attempts to signal the spleen to reduce the activation of T-cells and macrophages in the spleen. A recent study published by Rosas-Ballina et al [2001] indicated the existence of acetylcholine-synthesizing T-cells in the spleen that may respond to vagal stimulation, resulting, for example, in suppression of inflammatory response/TNF-alpha via macrophages.

It is expected that during the life of a patent maturing from this application many relevant technologies will be developed and the scope of the terms used herein is intended to include all such new technologies a priori. As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A tool for ablation of tissue in a living patient comprising:
a plurality of ablation electrodes;
a basket mounted axially to a shaft, said basket having:
a radially contracted configuration wherein supports of said basket are oriented along an axis of said basket for fitting into a channel of a catheter, a distal end of said catheter fitting into a lumen of the living patient; and
a radially spread configuration wherein said supports are spread radially away from said axis for holding said plurality of electrodes against an inner wall of said lumen;
a cup shaped embolic trap configured to spread to block said lumen to transport of emboli, said embolic trap spreading radially around an apex located along an axis of said basket and distal to said basket; and
a manipulation apparatus configured to be accessible from the proximal end of said catheter said manipulation apparatus configured for:
reversibly extending and retrieving said shaft including said basket and said plurality of electrodes and said embolic trap through a distal opening of said catheter; and
reversibly switching said basket between said radially contracted configuration and said radially spread configuration.

2. The ablation tool of claim 1, wherein said embolic trap is mounted to said shaft, distal of said basket.

3. The ablation tool of claim 1, wherein said embolic trap is mounted to a distal end of said basket.

4. The ablation tool of claim 1, wherein said plurality of ablation electrodes, said embolic trap and said basket fit concurrently into said channel.

5. The tool of claim 1, wherein a distance between said basket and said trap along the axis of said channel is fixed.

6. The tool of claim 1, wherein said embolic trap also has a radially spread and a radially contracted configuration and where said manipulation apparatus is further configured for reversibly switching said embolic trap between said a radially spread and a radially contracted configuration.

7. The tool of claim 6, wherein said basket is spread and contracted independently from said embolic trap.

8. The tool of claim 6, wherein said manipulation apparatus spreads said basket only when said embolic trap is in said radially spread configuration.

9. The tool of claim 1, wherein said basket and said embolic trap have three stages of deployment:
a fully retracted state wherein both said embolic trap and basket are radially contracted;
an intermediate state wherein said embolic trap radially spread and said basket is radially contracted; and
a fully expanded state wherein said embolic trap and basket are radially expended.

10. The tool of claim 1, further comprising:
one or more sensors configured to detect a slew rate and/or propagation time between two electrodes, said two electrodes being selected from said plurality of ablation electrodes and a dispersive electrode.

11. The tool of claim 1, further comprising:
a dispersive electrode having a surface area of electrical contact at least ten times the surface area of electrical contact of at least one electrode of said plurality of ablation electrodes.

12. The tool of claim 11, wherein a distal end of said dispersive electrode is located at least 5 mm proximal from the most proximal electrode of said plurality of ablation electrodes.

13. The tool of claim 11, wherein a distal end of said dispersive electrode is located less than 100 mm proximal from most proximal electrode of said plurality of ablation electrodes.

14. The tool of claim 1, further comprising:
an insulator electrically insulating at least one of said plurality of ablation electrodes from a fluid in said lumen.

15. The tool of claim 11, further comprising:
one or more sensors detecting an indicator of ablation progress; and
a control unit programmed to:
receive from said one or more sensors an indicator of progress of a bipolar ablation process between a pair of said plurality of ablation electrodes,
identify a zone for further ablation based on said received indicator, and
instruct to ablate said zone with a unipolar signal between said dispersive electrode and at least one of said plurality of ablation electrodes.

16. The ablation catheter of claim 15, wherein said one or more sensors detect a slew and/or propagation time between two electrodes selected from said plurality of ablation electrodes and said dispersive electrode.

17. A system for determining progress of denervation of a lumen located in a living patient, comprising:
a sheath, a distal end of said sheath for insertion into the lumen,
a plurality of ablation electrodes;
a basket mounted axially to a shaft, said basket having:
a radially contracted configuration wherein supports of said basket are oriented along an axis of said basket for fitting into a channel of a catheter, a distal end of said catheter fitting into the lumen; and
a radially spread configuration wherein said supports are spread radially away from said axis for holding said plurality of electrodes against an inner wall of the lumen;
a manipulation apparatus configured to be accessible from the proximal end of said catheter said manipulation apparatus configured for:
reversibly extending and retrieving said basket and said plurality of electrodes through a distal opening of said sheath; and
reversibly switching said basket between said radially contracted configuration and said radially spread configuration; and
a control unit configured to detect a parameter selected from the group consisting of a slew rate and propagation time between at least one pair of said plurality of ablations electrodes.

18. The system of claim 17, further comprising:
an embolic trap configured for blocking transport of emboli in said lumen and wherein said manipulation apparatus is further configured for
reversibly extending and retrieving said embolic trap through a distal opening of said sheath.

19. An ablation device comprising:
a plurality of pairs of ablation electrodes arranged along a single shaft;
said single shaft having at least two configurations,
a longitudinally stretched configuration wherein said plurality of pairs of ablation electrodes are arranged linearly for insertion into a channel of a catheter fitting into a lumen, and
a radially spread configuration wherein said single shaft is bent into a helix that is circumscribed by and in contact with an inner wall of said lumen and retains said plurality of pairs of ablation electrodes in a predetermined pattern along said inner wall of said lumen; and
a manipulation mechanism accessible from outside said lumen, said manipulation mechanism for longitudinally contracting said single shaft inside said lumen from said stretched configuration to said radially spread configuration.

20. The ablation device of claim 19, wherein a proximal end of said shaft is connected to a catheter extending out of said lumen.

21. The ablation device of claim 20, wherein a proximal end of said helix is centered along said lumen.

22. An ablation catheter comprising:
a stem including a junction at a distal end thereof;
a plurality of branches extending from said junction, each of said plurality of branches including a plurality of electrodes; and
a control unit configured for transmitting a radio frequency ablation signal between at least one of said plurality of electrodes of a first branch of said plurality of branches to at least one electrode of said plurality of electrodes on a second branch one of said plurality of branches.

23. The ablation catheter of claim 22, wherein at least one of said plurality of branches is retractable.

24. The ablation catheter of claim 22, wherein a distance between said junction and a distal end of at least one of said plurality of branches is between 10 to 50 mm from said junction.

25. The ablation catheter of claim 22, wherein a distance between said at least one electrode and said junction is between 3 to 20 mm.

26. The ablation catheter of claim 22, wherein a width of said stem is less than 9 Fr.

27. The ablation catheter of claim 22, wherein a width of said stem is less than 6 Fr.

28-38. (canceled)
